(19) United States
(12) Patent Application Publication
(10) Pub. No.: US 2011/0144358 A1
(43) Pub. Date: Jun. 16, 2011

(54) Process for the manufacture of trimethylhydroquinone dialkanoates

(75) Inventors: Werner Bonrath, Freiburg (DE); Yann Foricher, Strasbourg (FR)

(73) Assignee: DSM IP Assets B.V., Heerlen (NL)

(21) Appl. No.: 12/929,835

(22) Filed: Feb. 18, 2011

Related U.S. Application Data
(63) Continuation of application No. 10/582,672, filed on Jun. 29, 2006.

(30) Foreign Application Priority Data
Dec. 15, 2003 (EP) 03028811.2

(51) Int. Cl. C07D 311/04; C07D 311/00; C07C 39/10

(52) U.S. Cl. 549/410; 549/408; 568/763

(57) ABSTRACT

The present invention is directed to a process for the manufacture of a 2,3,5-trimethylhydroquinone dialkanoate comprising reacting ketoisophorone with an acylating agent in the presence of an indium salt as the catalyst. Preferred are indium(III) salts such as indium trichloride or indium tris (trifluoromethanesulfonate). Further aspects of the present invention are a process for the manufacture of 2,3,5-trimethylhydroquinone using 2,3,5-trimethylhydroquinone dialkanoate as the starting material, especially a process for the manufacture of 2,3,5-trimethylhydroquinone by transesterification of 2,3,5-trimethylhydroquinone dialkanoate, as well as a process for the manufacture of a-tocopherol and its alkanoates, especially of (all-rac)-a-tocopherol and its acetate, comprising the reaction of ketoisophorone to 2,3,5-trimethylhydroquinone dialkanoate according to the present invention. Furthermore, the present invention also deals with a process for the manufacture of formulations of a-tocopherol and its alkanoates, especially of formulations of (all-rac)-a-tocopherol and its acetate, comprising the reaction of ketoisophorone to 2,3,5-trimethylhydroquinone dialkanoate according to the present invention.

This application is a continuation of U.S. application Ser. No. 10/582,672, filed Jun. 29, 2006, which is the U.S. Pat. No. national phase of international application PCT/EP2004/013903 filed 7 Dec. 2004 which designated the U.S. and claims benefit of EP 03028811.2, dated 15 Dec. 2003, the entire content of which is hereby incorporated by reference.

The present invention is concerned with a process for the manufacture of 2,3,5-trimethylhydroquinone dialkanoates.

The products of the process are useful as reactants for the manufacture of 2,3,5-trimethylhydroquinone, itself a known valuable reactant for the manufacture of (all-rac)-α-tocopherol, the most active member of the vitamin E group.

2,3,5-Trimethylhydroquinone dialkanoates are known to be producible by reacting ketoisophorone with an acylating agent in the presence of a catalyst. Many such catalysts have been proposed in the past for this purpose, in particular protonic acids, e.g. inorganic acids as sulphuric acid; organic acids as p-toluenesulphonic acid; strongly acidic ion exchange resins; and Lewis acids as zinc chloride, boron trifluoride, antimony pentafluoride and titanium tetrachloride: see inter alia DE-OS 21 49 159, EP-A 0 916 642 and EP-A 1 028 103. The known procedures, depending on the particular catalyst used, entailed certain disadvantages such as cost and lack of stability of the catalyst, use of chlorinated compounds, unsatisfying yield and formation of by-products.

In accordance with the present invention it has been that the conversion of ketoisophorone to 2,3,5-trimethylhydroquinone dialkanoates can be advantageously accomplished by the use of an idium salt as a catalyst.

Accordingly, the present invention provides a process for the manufacture of a 2,3,5-trimethylhydroquinone dialkanoates, which process comprises reacting ketoisophorone with an acylating agent in the presence of an indium salt as a catalyst.

The indium salt used as the catalyst in the process of the present invention is suitably an indium(III) salt, preferably an indium(III) halide (e.g. indium trichloride, tribromide or triiodide) or indium(III) triflate [indium tris(trifluoromethansulfonate, In(SO₃CF₃)₃]. It may be present in an amount of from about 0.1 mol-% to about 2 mol-%, preferably in an amount of from about 0.1 mol-% to about 1 mol-%, based on the amount of ketoisophorone. The catalyst may be added to the reaction mixture in solid form, anhydrous or hydrated (of which InCl₃.4 H₂O is an example). It can also be used in solution or in suspension: Then the catalyst is dissolved or suspended in the organic solvent, in which the reaction may be carried out. The concentration of such a solution or suspension is not critical. Furthermore, the catalyst tolerates acetic anhydride and other acylating agents as well as traces of protonic solvents such as acetic acid, methanol, ethanol and water.

The acylating agent used in the process of the present invention may be any acylating agent that is conventionally used in the conversion of ketoisophorone to 2,3,5-trimethylhydroquinone dialkanoates, particularly acid anhydrides, acyl halides, and enol esters.

Examples of acid anhydrides are straight or branched chain alkanoic acid anhydrides such as acetic, propionic and butyric anhydride. Examples of acyl halides are straight or branched chain alkanoyl chlorides such as acetyl, propionyl and butyryl chloride. Finally, examples of enol esters are isopropenyl acetate and isopropenyl butyrate.

The preferred acylating agent is acetic anhydride or acetyl chloride, especially acetic anhydride.

The process of the present invention can be carried out in the presence or in the absence of a solvent. As a solvent, any inert polar or non-polar organic solvent or any mixture of two or more of such solvents can be used. In a preferred aspect of the invention, the reaction is carried out in the absence of a solvent.

Suitable classes of polar organic solvents include aliphatic and cyclic carbonates, aliphatic esters and cyclic esters (lactones), aliphatic and cyclic-ketones and mixtures thereof. Preferred examples of aliphatic and cyclic carbonates are ethylene carbonate, propylene carbonate and 1,2-butylene carbonate. Preferred examples of aliphatic esters and cyclic esters (lactones) are ethyl acetate, isopropyl acetate and n-butyl acetate; and γ-butyrolactone. Preferred examples of aliphatic and cyclic ketones are acetone, diethyl ketone and isobutyl methyl ketone; and cyclopentanone and isophorone.

As suitable classes of non-polar organic solvents there may be mentioned aliphatic hydrocarbons and aromatic hydrocarbons. Preferred examples of aliphatic hydrocarbons are linear, branched or cyclic C₅- to C₁₅-alkanes. Particularly preferred are linear, branched or cyclic C₆- to C₁₀-alkanes, especially preferred are hexane, heptane, octane, cyclohexane and methylcyclohexane or mixtures thereof. Preferred examples of aromatic hydrocarbons are benzene, toluene, o-, m- and p-xylene 1,2,3-trimethylbenzene, pseudocumene, mesitylen, naphthalene and mixtures thereof.

The reaction can be effected in a single solvent phase, two-phase solvent systems comprising polar and non-polar solvents may, however, also be used. Examples of non-polar solvents in such two-phase solvent systems are the non-polar solvents named above. Example of polar solvents in such two-phase solvent systems are the polar solvents named above. The most preferred two-phase solvent systems are mixtures of ethylene carbonate and/or propylene carbonate and hexane, heptane or octane, especially mixtures of ethylene carbonate and heptane, mixtures of propylene carbonate and octane, and mixtures of ethylene carbonate, propylene carbonate and heptane.

While the ratio of acylating agent to ketoisophorone is not narrowly critical the molar ratio of the acylating agent to ketoisophorone is suitably from about 1:1 to about 5:1, preferably from about 2:1 to about 3:1, and is most preferably about 3:1.

The process of the invention is conveniently carried out at temperatures from about 0° C. to about 140° C., preferably at temperatures from about 25° C. to about 90° C., more preferably at temperatures from about 25° C. to about 70° C.

Moreover, the process is conveniently carried out under an inert gas atmosphere, preferably under gaseous nitrogen or argon.

The progress of the reaction is suitably monitored by gas chromatography (GC) and mass spectrometry (MS) of samples taken from the reaction mixture at various time intervals during the reaction.

The produced 2,3,5-trimethylhydroquinone dialkanoate can be isolated after distilling off the remaining acylating agent and the secondary product formed in the acylation, e.g. acetic acid when acetic anhydride is used as the acylating agent, by extraction of the crude product mixture with a suitable organic solvent, e.g. toluene. Another isolation procedure is the crystallization of the 2,3,5-trimethylhydroquinone dialkanoate from the mixture at the termination of the reaction by cooling, and, optionally, adding water, to the mixture to promote the crystallization.

The catalyst can be recovered by extraction with water or acid-water and concentration of the extract. Alternatively, the catalyst can be recovered by adding a biphasic solvent system, e. g. a carbonate (particularly ethylene carbonate or propylene carbonate) and an aliphatic hydrocarbon (particularly heptane or octane), and isolating it from the polar (carbonate) phase.

### Process For the Manufacture of 2,3,5-Trimethylhydroquinone

The 2,3,5-trimethylhydroquinone dialkanoate obtained by the process of the present invention can be converted into 2,3,5-trimethylhydroquinone e.g. by transesterification, i.e. by treatment with an alcohol, e.g. an aliphatic alcohol such as isopropanol or n-butanol. Depending on the amounts of alcohol and catalyst and on the temperature in the reaction mixture, the transesterification yields the unesterified 2,3,5-trimethylhydroquinone and the ester formed as the further product. Other processes known to the person skilled in the art may also be used for producing 2,3,5-trimethylhydroquinone from 2,3,5-trimethylhydroquinone dialkanoate. Such in a further aspect the invention also deals with a process for the manufacture of 2,3,5-trimethylhydroquinone wherein the 2,3,5-trimethylhydroquinone dialkanoate obtained by the process of the present invention is used as starting material.

### Process For the Manufacture of α-Tocopherol Or Its Alkanoates

The former product, 2,3,5-trimethylhydroquinone, can be converted into e.g. (all-rac)-α-tocopherol by reaction with isophytol (and/or phytol), preferably in a biphasic solvent system, e.g. in a solvent system comprising a polar solvent such as ethylene or propylene carbonate, and an non-polar solvent, particularly an aliphatic hydrocarbon such as heptane. Other processes for the manufacture of α-tocopherol using 2,3,5-trimethylhydroquinone dialkanoate, 2,3,5-trimethylhydroquinone monoalkanoate or 2,3,5-trimethylhydroquinone as the intermediate or starting material are e.g. disclosed in U.S. Pat. No. 4,808,736, EP-A 0 257 503, EP-A 0 269 009, U.S. Pat. No. 5,283,346, U.S. Pat. No. 4,208,334, EP-A 0 012 824, EP-A 0 782 993, U.S. Pat. No. 5,900,494, WO 98/21197, U.S. Pat. No. 5,908,939, EP-A 1 000 940, U.S. Pat. No. 6,423,851, EP-A 1 134 218, U.S. Pat. No. 6,369,242, WO 2004/046127 and WO 2004/063182. Thus, in a further aspect the invention deals with a process for the manufacture of α-tocopherol or its alkanoates, especially with a process for the manufacture of (all-rac)-α-tocopherol and/or its acetate, wherein 2,3,5-trimethylhydroquinone dialkanoate obtained according to the process of the present invention is used as intermediate or starting material.

### Process For the Manufacture of Formulations of α-Tocopherol Or Its Alkanoates

The α-tocopherol or its alkanoate obtained by one of the processes disclosed in the documents mentioned above using a trimethylhydroquinone dialkanoate obtained by a process according to the present invention as intermediate or starting material can further be formulated by any method known to the person skilled in the art, e.g. as those disclosed in U.S. Pat. No. 6,162,474, U.S. 2001/0009679, U.S. Pat. No. 6,180,130, U.S. Pat. No. 6,426,078, U.S. Pat. No. 6,030,645, U.S. Pat. No. 6,150,086, U.S. Pat. No. 6,146,825, U.S. Pat. No. 6,001,554, U.S. Pat. No. 5,938,990, U.S. Pat. No. 6,530,684, U.S. Pat. No. 6,536,940, U.S. 2004/0053372, U.S. Pat. No. 5,668,183, U.S. Pat. No. 5,891,907, U.S. Pat. No. 5,350,773, U.S. Pat. No. 6,020,003, U.S. Pat. No. 6,329,423, WO 96/32949, U.S. Pat. No. 5,234,695, WO 00/27362, EP 0 664 116, U.S. 2002/0127303, U.S. Pat. No. 5,478,569, U.S. Pat. No. 5,925,381, U.S. Pat. No. 6,651,898, U.S. Pat. No. 6,358,301, U.S. Pat. No. 6,444,227, WO 96/01103 and WO 98/15195. Therefore, the present invention is also directed to a process for the manufacture of formulations of α-tocopherol and its alkanoates, especially of formulations of (all-rac)-α-tocopherol and its acetate, comprising the reaction of ketoisophorone to 2,3,5-trimethylhydroquinone dialkanoate according to the present invention as described above.

The invention is illustrated by the following Example.

### EXAMPLE

A 230-ml four-necked flat-bottomed flask with heating/cooling jacket, equipped with a thermometer, a glass-tube for Ar-purge, a reflux condenser and a mechanical stirrer, was filled with the amount of catalyst given in the table below and 20.65 g (133 mmol) of ketoisophorone and a yellow solution was obtained. Within 10 minutes 40.64 g (400 mmol, 37.60 ml) of acetic anhydride were added under stirring. During addition, the mixture turned dark yellow to finally dark brown. The internal temperature was regulated by a thermostat. Samples were withdrawn and submitted to qualitative GC-analysis. The results are tabulated below:

| Catalyst | Temper- | Reaction | | Purity [%] | | |
|---|---|---|---|---|---|---|
| (amount) | ature | time | Conversion | | TMHQ- | TMC- |
| [mol %] | [° C.] | [hours] | [%] | KIP | DA | DA |
| InCl₃ (1) | 25 | 18 | 23.9 | 76.1 | 7.2 | 1.6 |
| InCl₃ (1) | 50 | 21 | 45.5 | 54.6 | 37.2 | 5.8 |
| InCl₃ (1) | 50 | 37^{a} | 50.0 | 50.0 | 42.3 | 6.6 |
| InCl₃ (2 + 1)^{b} | 50 | 76^{b} | 78.9 | 21.1 | 68.3 | 10.0 |
| In(SO₃CF₃)₃ | 50 | 5 | 100.0 | 0.0 | 93.5 | 6.8 |
| (1) | | | | | | |
| In(SO₃CF₃)₃^{a} | 50 | 10^{a} | 65.3 | 34.7 | 58.5 | 4.1 |
| (0.1) | | | | | | |

KIP = ketoisophorone;
TMHQ-DA = 2,3,6-trimethylhydroquinone diacetate;
TMC-DA = 2,3,6-trimethyl catechol diacetate.
The molar ratio of Ac₂O/KIP was 3/1.
The amount of catalyst is based on KIP.
^{a}Inhibition of the reaction after the indicated time.
^{b}1 mol % of catalyst was added after 76 hours as inhibition of the reaction occurred but no progress was observed after this addition.

1. A process for the manufacture 2,3,5-trimethylhydroquinone dialkanoate comprising reacting ketoisophorone with an acylating agent in the presence of an indium salt as a catalyst.

2. The process according to claim 1, wherein the indium salt is indium trichloride or indium tris (trifluoromethanesulfonate).

3. The process according to claim 1, wherein the acylating agent is an acid anhydride, an acyl halide or an enol ester.

4. The process according to claim 3, wherein the acylating agent is a straight or branched chain alkanoic acid anhydride, preferably acetic, propionic or butyric anhydride; a straight or branched chain alkanoyl chloride, preferably acetyl, propionyl or butyryl chloride; or, an enol ester, preferably isopropenyl acetate or butyrate.

5. The process according to claim 1, wherein the molar ratio of the acylating agent to ketoisophorone is from about 1:1 to about 5:1, preferably from about 2:1 to about 3:1, most preferably about 3:1.

6. The process according to claim 1, wherein the amount of the indium salt used as the catalyst is from about 0.1 mol-% to about 2 mol-%, preferably from about 0.1 to about 1 mol-%, based on the amount of ketoisophorone.

7. The process according to claim 1, wherein the acylating reaction is carried out at a temperature of from about 0° C. to about 140° C., preferably from about 25° C. to about 90° C., more preferably from about 25° C. to about 70° C.

8. The process according to claim 1, wherein the 2,3,5-trimethylhydroquinone dialkanoate obtained is converted into (all-rac)-α-tocopherol by transesterification to yield 2,3,5-trimethylhydroquinone and reaction of the latter with isophytol and/or phytol.

9. A process for the manufacture of 2,3,5-trimethylhydroquinone whereby the 2,3,5-trimethylhydroquinone dialkanoate obtained according to claim 1 is used as starting material.

10. The process according to claim 9, whereby the 2,3,5-trimethylhydroquinone dialkanoate is transesterified to 2,3,5-trimethylhydroquinone.

11. A process for the manufacture of α-tocopherol and its alkanoates, especially of (all-rac)-α-tocopherol and its acetate, comprising the reaction of ketoisophorone to 2,3,5-trimethylhydroquinone dialkanoate according to claim 1.

12. A process for the manufacture of formulations of α-tocopherol and its alkanoates, especially of formulations of (all-rac)-α-tocopherol and its acetate, comprising the reaction of ketoisophorone to 2,3,5-trimethylhydroquinone dialkanoate according to claim 1.
